(19) United States
(12) Patent Application Publication
(10) Pub. No.: US 2025/0115837 A1
(43) Pub. Date: Apr. 10, 2025

(54) Bioengineered Models Of Lung Development

(71) Applicant: The Trustees of the University of Pennsylvania, Philadelphlia, PA (US)

(72) Inventors: Dongeun Huh, Villanova, PA (US); Sunghee Estelle Park, Philadelphia, PA (US)

(21) Appl. No.: 18/911,632

(22) Filed: Oct. 10, 2024

Related U.S. Application Data
(60) Provisional application No. 63/589,245, filed on Oct. 10, 2023.

(51) Int. Cl. C12M 3/00; C12M 1/00; C12M 3/06; C12N 5/071

(52) U.S. Cl. CPC C12M 21/08; C12M 23/16; C12M 23/26; C12M 23/34; C12N 5/0688; C12N 2513/00

(57) ABSTRACT

A lung-on-a-chip microfluidic chip, comprising: a first region, the first region comprising a central channel and at least one side channel adjacent thereto, the central channel having therein a plurality of cells disposed in a matrix; and a second region, the second region comprising a vacuum chamber; and a membrane disposed between the central channel and the vacuum chamber, the membrane arranged so as to maintain fluidic isolation between the central channel and the vacuum chamber, and the membrane arranged such that a pressure within the vacuum chamber effects movement of the membrane such that the plurality of cells disposed in the matrix experience a mechanical force related to the movement of the membrane. A method, comprising changing a pressure within the vacuum chamber of a microfluidic chip according to the present disclosure.

### RELATED APPLICATION

This application claims benefit to U.S. Provisional Application No. 63/589,245, filed Oct. 10, 2023, the entirety of which is incorporated by reference herein.

### TECHNICAL FIELD

The present disclosure relates to the field of organ-on-a-chip devices.

### BACKGROUND

Lung development is a complex process of organogenesis that involves highly coordinated, dynamic changes in the structural organization and mechanical environment of developing tissues. As a unique signature of this dynamic biomechanical process, the fetal breathing movements (FBMs) occur when contracting respiratory muscles cause the fetus to swallow and expel amniotic fluid. FBMs are detected as early as 10-15 weeks of gestation and are known to occur more frequently and to a greater extent with the progression of pregnancy. Studies have suggested that mechanical forces generated by FBMs may have significant effects on fetal lung tissues but research to date has focused predominantly on the early process of lung development. As a result, our understanding remains rudimentary as to how FBM-induced forces influence the later stages of lung development, especially the formation and maturation of alveolar compartments that play a central role in the specialized function of the respiratory system.

### SUMMARY

To address this gap, we developed a microengineered organoid-on-a-chip platform for investigating alveolar development in the human lung. Our approach leverages human pluripotent stem cell-derived lung organoids that follow the developmental trajectories of distal lung in vitro and differentiate into human alveolar epithelial cells. By growing these organoids in a mechanically actuatable microdevice, our microengineered model enables the generation and controlled application of physiologically-relevant mechanical forces to developing alveolar organoids to recapitulate human fetal lungs experiencing FBMs. Using this system, our study reveals striking effects of FBM-induced forces on morphogenesis and maturation of human lung tissues.

In meeting the described long-felt needs, the present disclosure provides a lung-on-a-chip microfluidic chip, comprising: a first region, the first region comprising a central channel and at least one side channel adjacent thereto, the central channel having therein a plurality of cells disposed in a matrix; and a second region, the second region comprising a vacuum chamber; and a membrane disposed between the central channel and the vacuum chamber, the membrane arranged so as to maintain fluidic isolation between the central channel and the vacuum chamber, and the membrane arranged such that a pressure within the vacuum chamber effects movement of the membrane such that the plurality of cells disposed in the matrix experience a mechanical force related to the movement of the membrane.

Also provided is a method, comprising changing a pressure within the vacuum chamber of a microfluidic chip according to the present disclosure.

### BRIEF DESCRIPTION OF THE DRAWINGS

The patent or application file contains a least one drawing/photograph executed in color. Copies of this patent or patent application publication with color drawing(s)/photograph(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1C depict a chip according to the present disclosure, showing microengineered alveologenesis-on-a-chip.

FIGS. 2A-2F provide exemplary images of the effects of FBM-like mechanical forces on alveolar development.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

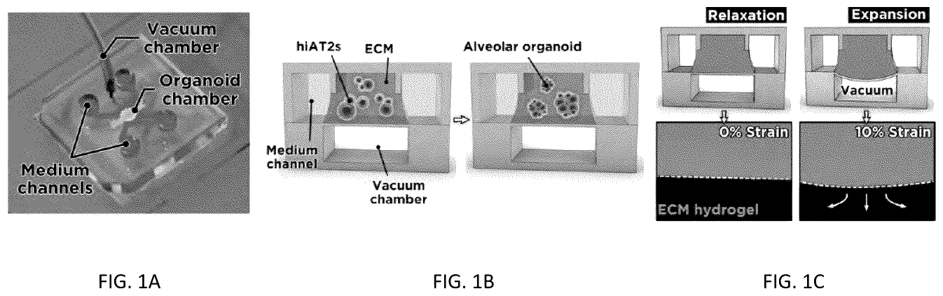

A characteristic of the lung organoids-on-a-chip system developed in our study is its capability to dynamically deform an extracellular matrix (ECM) hydrogel scaffold containing developing human lung organoids. This device is made of poly(dimethylsiloxane) and includes an organoid culture chamber flanked by two side microchannels for nutrient supply and a mechanically actuatable elastomeric membrane controlled by a pneumatic chamber (FIG. 1A). To establish alveolar organoid culture in this device, we inject a mixture of human pluripotent stem cell-derived alveolar type 2 (AT2) cells and a hydrogel precursor solution into the organoid culture chamber (FIG. 1B). After the formation of the organoid-containing hydrogel scaffold, alveolar organoid culture medium is introduced into the side channels to provide nutrients to the embedded organoids. During culture, the hAT2 cells in the ECM scaffold proliferate and self-assemble to form alveolar organoids (FIG. 1B). Importantly, this microengineered platform allows for production and precise control of dynamic mechanical forces reminiscent of FBMs through on-demand application of vacuum to the bottom chamber sealed against a thin PDMS membrane (FIG. 1C). In this setup, organoids are cultured for at least 30 days in either static or dynamic conditions and then harvested and fixed for subsequent analysis.

### Results/Conclusions/Discussions

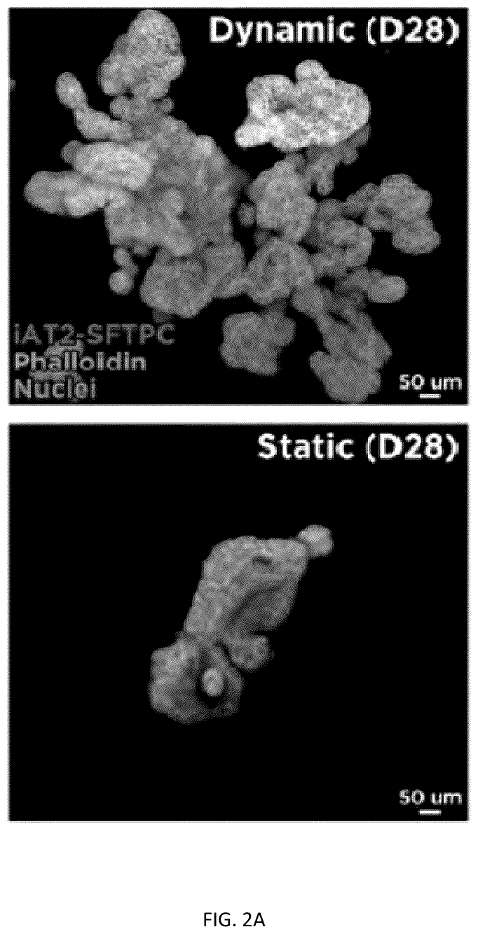

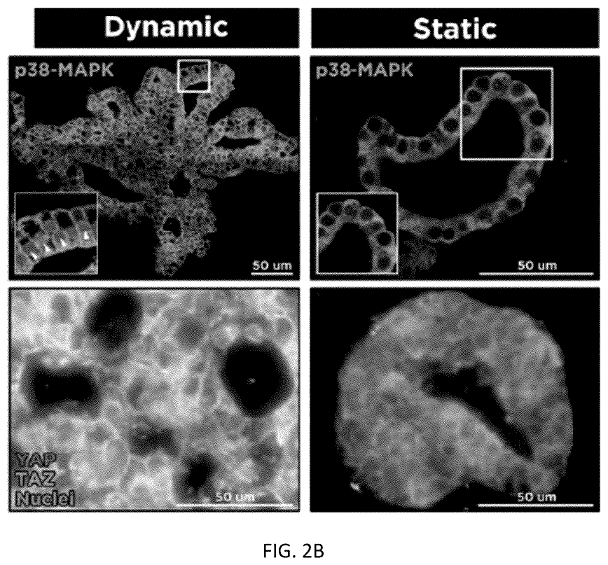

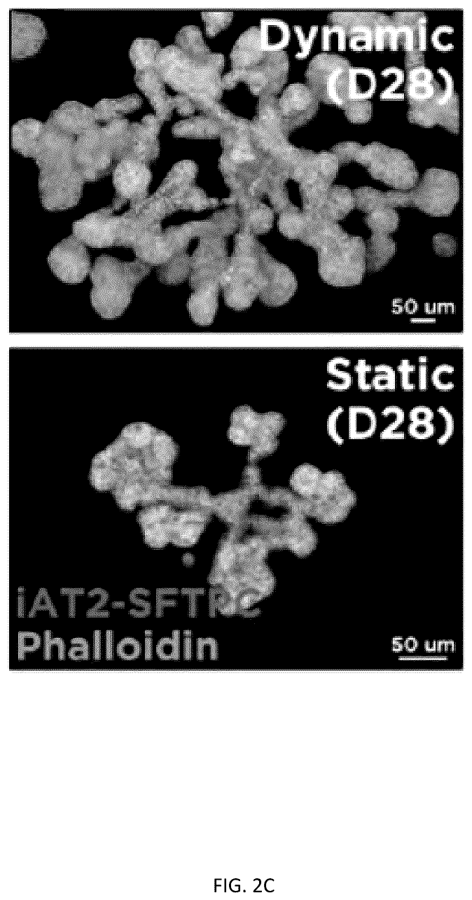

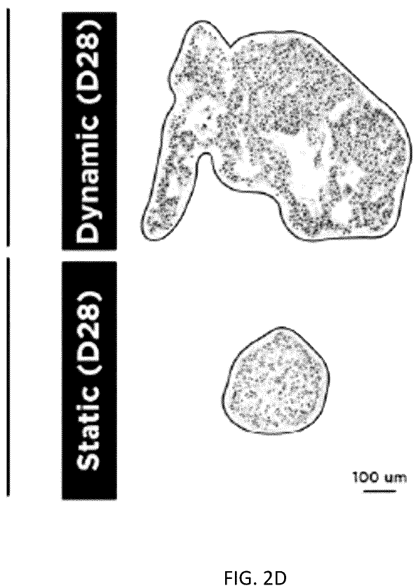

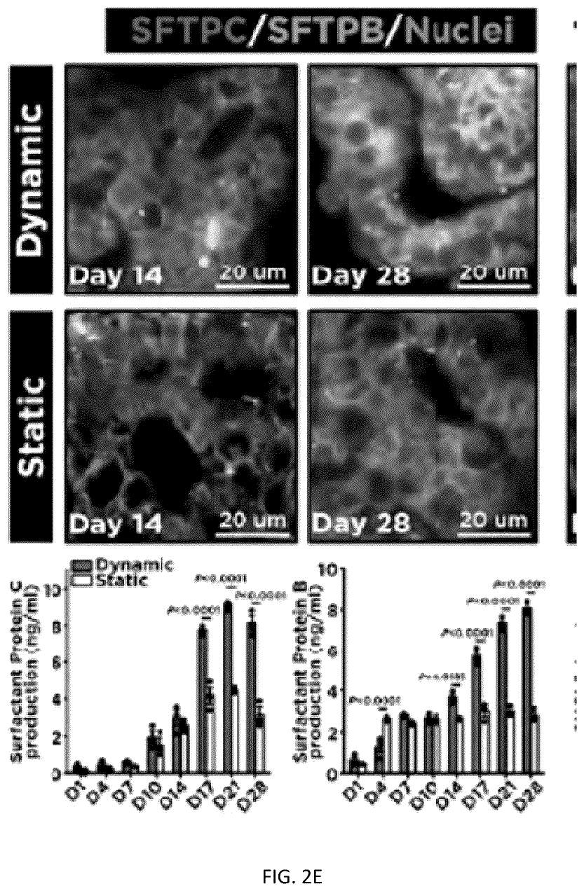

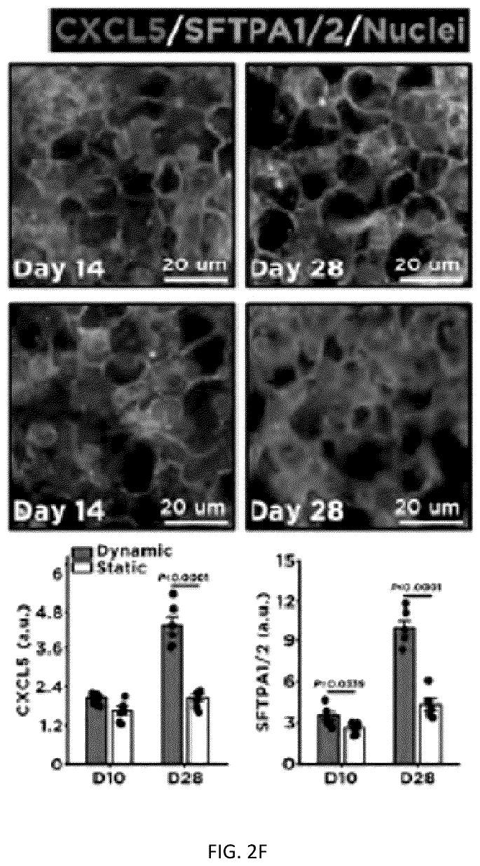

We first examined whether our system supports extended development of alveolar organoids by culturing hPSC-derived NKX2-1+/SFTPC+AT2 cells in Matrigel. hAT2s proliferated and formed alveolar organoids within the first 10 days of culture. Interestingly, organoids stimulated with FBM-like mechanical forces from day 10 exhibited significantly increased size and number of buds, whereas organoids maintained in static conditions showed arrested growth and disintegrated after 28 days (FIG. 2A). We also analyzed the activation of mechanoresponsive and mechanotransduction signaling pathways through immunocytochemical analysis and found that mechanical stimulation allowed the organoids to express significantly higher levels of MAPK and YAP/TAZ expression compared to those in static culture (FIG. 2B).

The data revealed that FBM-like mechanical forces affect the architecture of developing alveolar organoids over time, leading to extensive epithelial growth and branching morphology (FIG. 2C). During long-term culture for over a month, we observed a more than 6-fold increase in size, accompanied by the formation of large numbers of aerated space structures that resemble development of alveolar septa (FIG. 2D). In addition, the dynamic culture environment in this model led to significantly enhanced alveolar epithelial differentiation and maturation during organoid development (FIGS. 2E, 2F). Using advanced capabilities to mimic the dynamic mechanical environment of the fetal lung, our work shows how mechanical forces regulate a complex developmental program of alveolar epithelial cell differentiation and maturation at late stages of lung development.

### Aspects

The following Aspects are illustrative only and do not limit the scope of the present disclosure or the appended claims. Any part or parts of any one or more Aspects can be combined with any part or parts of any one or more other Aspects.

Aspect 1. A lung-on-a-chip microfluidic chip, comprising: a first region, the first region comprising a central channel and at least one side channel adjacent thereto, the central channel having therein a plurality of cells disposed in a matrix; and a second region, the second region comprising a vacuum chamber; and a membrane disposed between the central channel and the vacuum chamber, the membrane arranged so as to maintain fluidic isolation between the central channel and the vacuum chamber, and the membrane arranged such that a pressure within the vacuum chamber effects movement of the membrane such that the plurality of cells disposed in the matrix experience a mechanical force related to the movement of the membrane.

Aspect 2. The microfluidic chip of Aspect 1, wherein the matrix comprises a hydrogel.

Aspect 3. The microfluidic chip of any one of Aspects 1-2, wherein the plurality of cells comprises an organoid, the organoid optionally comprising an alveolar organoid.

Aspect 4. The microfluidic chip of any one of Aspects 1-2, wherein the plurality of cells comprises human pluripotent stem cell-derived alveolar type 2 cells.

Aspect 5. The microfluidic chip of any one of Aspects 1-4, wherein the at least one side channel comprises a culture medium therein.

Aspect 6. A method, comprising changing a pressure within the vacuum chamber of a microfluidic chip according to any one of Aspects 1-5.

Aspect 7. The method of Aspect 6, wherein the changing is according to a force schedule.

Aspect 8. The method of Aspect 7, wherein the force schedule is in accordance with physiological movements.

Aspect 9. The method of Aspect 8, wherein the force schedule simulates fetal breathing movements.

Aspect 10. The method of any one of Aspects 6-9, wherein the schedule gives rise to the plurality of cells substantially recapitulating a physiological tissue, the physiological tissue optionally being a physiological tissue that experiences the force schedule in vivo.

What is claimed:

1. A lung-on-a-chip microfluidic chip, comprising:
a first region,
the first region comprising a central channel and at least one side channel adjacent thereto,
the central channel having therein a plurality of cells disposed in a matrix; and
a second region,
the second region comprising a vacuum chamber; and
a membrane disposed between the central channel and the vacuum chamber,
the membrane arranged so as to maintain fluidic isolation between the central channel and the vacuum chamber, and
the membrane arranged such that a pressure within the vacuum chamber effects movement of the membrane such that the plurality of cells disposed in the matrix experience a mechanical force related to the movement of the membrane.

2. The microfluidic chip of claim 1, wherein the matrix comprises a hydrogel.

3. The microfluidic chip of claim 1, wherein the plurality of cells comprises an organoid, the organoid optionally comprising an alveolar organoid.

4. The microfluidic chip of claim 1, wherein the plurality of cells comprises human pluripotent stem cell-derived alveolar type 2 cells.

5. The microfluidic chip of claim 1, wherein the at least one side channel comprises a culture medium therein.

6. A method, comprising changing a pressure within the vacuum chamber of a microfluidic chip according to claim 1.

7. The method of claim 6, wherein the changing is according to a force schedule.

8. The method of claim 7, wherein the force schedule is in accordance with physiological movements.

9. The method of claim 8, wherein the force schedule simulates fetal breathing movements.

10. The method of claim 6, wherein the schedule gives rise to the plurality of cells substantially recapitulating a physiological tissue, the physiological tissue optionally being a physiological tissue that experiences the force schedule in vivo.
